(19) United States
(12) Patent Application Publication
(10) Pub. No.: US 2007/0184126 A1
(43) Pub. Date: Aug. 9, 2007

(54) Water-soluble bound matter of proanthocyandin and composition containing the same

(75) Inventors: Kinya Takagaki, Fukuoka (JP); Sadao Mori, Fukuoka (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: TOYO SHINYAKU CO., LTD., Fukuoka-shi, Fukuoka (JP)

(21) Appl. No.: 10/598,858

(22) PCT Filed: Dec. 24, 2004
(86) PCT No.: PCT/JP04/19793

§ 371 Date: Sep. 13, 2006

(30) Foreign Application Priority Data
Mar. 15, 2004 (JP) 2004-72382

(51) Int. Cl. A61K 36/18; A61K 38/16; A61K 38/14

(52) U.S. Cl. 424/725; 514/8

(57) ABSTRACT

The present invention provides a water-soluble conjugate consisting of proanthocyanidins and a peptide containing three or more amino acids. Preferably, the average molecular weight of the peptide is not more than 7,000. The water-soluble conjugate of the present invention provides proanthocyanidins having a high protective stability and retained or increased bioactivities. Namely, it does not cause precipitation even in a long-term storage and can be transported into the body while maintaining the bioactivities.

### TECHNICAL FIELD

The present invention relates to a water-soluble conjugate (water-soluble bound matter) for stabilizing proanthocyanidins and a composition containing the same.

### BACKGROUND ART

Proanthocyanidins, which are condensed tannins that are condensation polymers having flavan-3-ol and/or flavan-3,4-diol as a constituent unit and having a degree of polymerization of 2 or more, have been used for a long time for the purpose of obtaining effects of conditioning the skin such as an astringent effect on the skin. Having a variety of activities, such as antioxidative properties and a whitening effect, proanthocyanidins have been recently used for food products, cosmetics, and the like (Japanese Laid-Open Patent Publication Nos. 61-16982 and 2-134309). For example, proanthocyanidins have been also used for cosmetics containing a protein (Japanese Laid-Open Patent Publication Nos. 11-75708, 6-336423, and 2002-238497).

However, since proanthocyanidins have a high antioxidation ability and hence are susceptible to oxidation, they undergo autoxidative polymerization. Accordingly, there are problems in that, for example, proanthocyanidins are colored easily. Furthermore, since the degree of polymerization of proanthocyanidins increases due to the autoxidative polymerization, the solubility of proanthocyanidins in water decreases, and thus precipitation tends to occur. There is also a problem in that the absorption efficiency of such proanthocyanidins having a high degree of polymerization into the body is decreased.

Japanese Laid-Open Patent Publication No. 2001-270881 describes a process for preventing discoloration of proanthocyanidins in a solution due to the oxidative polymerization by adding an amino acid having a hydroxyl group or a dipeptide containing that amino acid to proanthocyanidins. However, with a combination thereof components, precipitation occurs in a long-term storage in a liquid state.

Furthermore, there is another problem in that also when proanthocyanidins are decomposed by the digestion process or the like in the body, proanthocyanidins can no longer retain a state where their activities are maintained, resulting in a decreased absorption efficiency.

### DISCLOSURE OF INVENTION

It is an object of the present invention to provide proanthocyanidins with a high stability and having retained or increased bioactivities, that is to say, proanthocyanidins that do not cause precipitation even in a long-term storage and that can be transported into the body while maintaining the bioactivities.

As a result of in-depth studies on the above-described problems, the inventors of the present invention found a remarkable fact that a water-soluble conjugate of proanthocyanidins that does not cause precipitation even in a long-term storage, that has superior bioactivities, and that can be transported into the body while maintaining the bioactivities can be obtained by binding a particular peptide to proanthocyanidins, and thus, the present invention was achieved.

The water-soluble conjugate of the present invention consists of a proanthocyanidin and a peptide containing three or more amino acids.

In a preferred embodiment, the average molecular weight of the peptide is not more than 7,000.

In a preferred embodiment, the proanthocyanidin is derived from a plant extract.

The composition of the present invention contains the above-described water-soluble conjugate.

According to the present invention, especially when the proanthocyanidins from a plant extract is used, water-soluble conjugate of proanthocyanidins and a peptide containing three or more amino acids has a superior protective stability of proanthocyanidins, has a property of being absorbed well, does not cause precipitation even in a long-term storage, and has bioactivities due to proanthocyanidins. Furthermore, since the proanthocyanidins in the water-soluble conjugate are not decomposed even in the processes of digestion and absorption, the bioactivities are retained even in the body.

### BEST MODE FOR CARRYING OUT THE INVENTION

Hereinafter, the water-soluble conjugate of proanthocyanidins and a peptide containing three or more amino acids, and the composition containing the water-soluble conjugate of the present invention will be described. The present invention is not limited to construction described below, and it will be apparent to those skilled in the art that various modifications can be made to the present invention within the scope of the spirit of the invention.

(Proanthocyanidins)

Proanthocyanidins used in the water-soluble conjugate of the present invention refer to a group of compounds that are condensation products having flavan-3-ol and/or flavan-3,4-diol as a constituent unit and having a degree of polymerization of 2 or more. Proanthocyanidins are one type of polyphenols, and potent antioxidants produced by plants. Proanthocyanidins described above are contained in the bark of pine, oak, bayberry, and the like; the fruit or seeds of grape, blueberry, strawberry, avocado, locust, cowberry, and the like; the hull of barley, wheat, soybean, black soybean, cacao, adzuki bean, conker; the inner skin of peanuts; and the leaves of ginkgo, for example. Moreover, it is known that proanthocyanidins are also contained in cola nuts in West Africa, the roots of Rathania in Peru, and Japanese green tea.

As proanthocyanidins used in the present invention, condensation products having a lower degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer) are even more preferable. In this specification, condensation products having a degree of polymerization of 2 to 4 are referred to as oligomeric proanthocyanidins (OPCs). OPCs cannot be produced in the human body. In particular, although OPCs have strong binding ability to peptides, a conjugate of an OPC and a peptide does not cause precipitation and suspension in a solution, is stable in a powder or in a solution, and is preferred in that the conjugate has higher bioactivity than those of each component.

Proanthocyanidins described above are preferred to contain OPCs. Proanthocyanidins having a degree of polymerization of 5 or more is liable to cause suspension and precipitation by binding with peptides, however, when OPCs are contained in proanthocyanidins having a degree of polymerization of 5 or more, suspension and precipitation due to aggregation of proanthocyanidins having a degree of polymerization of 5 or more is decreased. There is no particular limitation regarding the ratio of proanthocyanidins having a degree of polymerization of 5 or more and OPCs. In view of suspension and precipitation due to aggregation with peptides, OPCs are contained in a ratio of preferably 1 part by weight with respect to 1 part by weight of proanthocyanidins having a degree of polymerization of 5 or more.

In the present invention, proanthocyanidins derived from plants is preferably used. In particular, extracts from plants containing OPCs, for example, extracts from barks, fruits or seeds are preferably used. In particular, extracts from plants that are rich in OPCs, for example, extracts containing at least 20 wt % of OPCs, preferably at least 30 wt %, more preferably at least 40% is used. Example of extracts from plants containing at least 20 wt % of OPCs includes a pine bark extract.

Since proanthocyanidins, in particular OPCs, are antioxidants as described above, they are known to provide an effect of reducing the possibility of adult diseases, such as cancer, cardiac diseases, cerebral thrombosis, and an effect of improving allergic diathesis, such as arthritis, atopic dermatitis, and pollenosis, and the like.

Furthermore, it is known that in addition to the antioxidation effect, OPCs also provide, for example, an effect of inhibiting bacterial proliferation in the oral cavity to reduce plaque (dental plaque); an effect of recovering the elasticity of blood vessels; an effect of improving the skin properties; an effect of enhancing collagen; an effect of improving hyperlipemia; an effect of preventing lipoprotein in blood from being damaged by active oxygen, thereby preventing aggregation and adherence of the oxidized fats onto the inside wall of the vessel, thus preventing cholesterol from being aggregated; an effect of regenerating vitamin E that has been degraded by active oxygen; and an effect of serving as an enhancer of vitamin E. Among these, by virtue of the effect of recovering the elasticity of blood vessels, blood flow is improved. Furthermore, when collagen is used as a peptide, by virtue of synergic effect with the effect of enhancing collagen, the skin properties are also improved.

When the plant extracts containing proanthocyanidins are used, it is preferable that catechins are contained in the plant extracts. Preferably, catechins are contained in the plant extracts in a ratio of 5 wt % or more. Catechins can be contained in the above-described plant extracts that are rich in proanthocyanidins. The term “Catechins” is a general term referring to polyhydroxyflavan-3-ols. As the catechins, for example, (+)-catechin, (−)-epicatechin, (+)-gallocatechin, (−)-epigallocatechin, epigallocatechin gallate, and epicatechin gallate are known. Gallocatechin, afzelechin, and 3-galloyl derivatives of (+)-catechin or gallocatechin are isolated from plant extracts, in addition to (+)-catechin that is called catechin in a narrow sense.

It is preferable that catechins are contained in a plant extract in a ratio of 0.1 parts by weight or more with respect to 1 part by weight of proanthocyanidins. More preferably, it is preferable that catechins are contained in a ratio of 5 wt % or more in a plant extract containing at least 20 wt % of OPCs. For example, when the catechin content in a pine bark extract is less than 5 wt %, it is possible to add catechins so that the catechin content becomes at least 5 wt %. It is most preferable to use a pine bark extract containing at least 5 wt % of catechins, at least 20 wt % of OPCs.

Catechins are known to have a cancer inhibiting ability, an arteriosclerosis preventing ability, a lipid metabolism disorder inhibiting ability, a blood pressure elevation inhibiting ability, a platelet aggregation inhibiting ability, an antiallergic ability, an antiviral ability, an antibacterial ability, a dental caries preventing ability, a halitosis preventing ability, an intestinal flora normalization ability, an active oxygen or free radical eliminating ability, an antioxidation ability, and the like. Catechins are known to have an antidiabetic ability to inhibit an elevation of blood glucose. Catechins have an effect of reducing aggregation/precipitation and suspension of proanthocyanidins. Catechins can also stabilize the bonding of OPCs and peptides, thereby high physiological activities are exhibited. Furthermore, the solubility of catechins in water is increased in the presence of OPCs, and catechins have an ability to activate the OPCs. Therefore, when catechins are ingested together with OPCs, catechins enhance effects of OPCs.

Hereinafter, a method for preparing proanthocyanidins will be described taking a pine bark extract that contains OPCs abundantly as an example.

As a pine bark extract, an extract from the bark of plant belonging to Pinales, such as French maritime pine (Pinus Martima), Larix Leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, are preferably used. Among these, French maritime pine (Pinus Martima) bark extract is preferable.

French maritime pine refers to maritime pines that grow in a part of the Atlantic coastal area in southern France. The bark of this French maritime pine contains proanthocyanidins, organic acids, and other bioactive substances, and the like, and it is known that proanthocyanidins, which are the main component, have a potent antioxidation effect of removing active oxygen.

The pine bark extract is obtained by extracting the bark of the above-described pines with water or an organic solvent. When water is used, warm water or hot water can be preferably employed. In view of improving the extraction efficiency, the water may preferably contain a salt such as sodium chloride. When an organic solvent is used, a solvent that is acceptable for production of food products or pharmaceuticals can be employed. Examples of such an organic solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. These water and organic solvents may be used alone or in combination of two or more. In particular, water, hot water, ethanol, aqueous ethanol, and aqueous propylene glycol are preferably used. In view of the safety when used in food products or pharmaceuticals, water, hot water, ethanol, and aqueous ethanol are more preferable.

There is no particular limitation on the method for extracting proanthocyanidins from pine bark, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation based on an adsorbent or absorbent.

Moreover, it is also possible to perform supercritical fluid extraction in which an entrainer is added. In this method, for example, about 2 to 20 W/V % of ethanol, propanol, n-hexane, acetone, toluene, or another aliphatic lower alcohol, aliphatic hydrocarbon, aromatic hydrocarbon, or ketone is added to the above-described fluid capable of forming a supercritical fluid, and the resultant fluid is turned to a supercritical fluid state and used to extract a target substance. With this method, the solubility of a target substance to be extracted, such as OPCs and catechins, in the extracting solvent can be dramatically increased, or the selectivity of separation can be enhanced. Thus a pine bark extract can be obtained efficiently.

It is also possible to employ a combination of a plurality of extraction processes to perform extraction from pine bark. By combining a plurality of extraction processes, pine bark extracts with various components can be obtained.

It is also possible to increase proanthocyanidin content of the above-described pine bark extract by purification. For purification, solvents such as ethyl acetate are commonly used. In view of the safety for food products or pharmaceuticals, it is preferable to purify the above-described pine bark extract using ethanol or water as a solvent by ultrafiltration or by a column chromatography or a batch method using an adsorptive carrier (e.g., DIAION HP-20, Sephadex-LH20).

The pine bark extract that is used as the plant extract of the present invention can be prepared using the following method. However, this method is merely an example and the present invention is not limited to this method.

First, 1 kg of the bark of French maritime pine is immersed in 3 L of a saturated aqueous solution of sodium chloride, and extraction is performed for 30 minutes at 100° C. to obtain an extract (extraction step). Then, the extract is filtrated, and the resultant insoluble material is washed with 500 mL of a saturated solution of sodium chloride to obtain a washed liquid (washing step). The extract and the washed liquid are combined to obtain a crude extract of pine bark.

Next, 250 mL of ethyl acetate are added to this crude extract, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of 1/5 of the original filtrated extract. The concentrated ethyl acetate extract is poured into 2 L of chloroform and stirred, and the resultant precipitate is recovered by filtration. Subsequently, this precipitate is dissolved in 100 mL of ethyl acetate, and then the resultant solution is added to 1 L of chloroform to form a precipitate. This process is repeated twice for washing. With this method, for example, about 5 g of a pine bark extract containing at least 20 wt % of OPCs and at least 5 wt % of catechins can be obtained.

Extracts from the above-described raw material plants, in particular, pine bark extracts described above, preferably contain at least 20 wt % of OPCs, more preferably at least 30 wt % expressed in terms of dry weight. Thus, as a raw material containing OPCs at a high rate, pine bark extract is preferably used

It should be noted that an extract extracted from a plant using water or ethanol as described above also contains proanthocyanidins having a degree of polymerization of 5 or more, but most of those proanthocyanidins have a degree of polymerization of not more than 10 to 20 because of the solubility of proanthocyanidins in a polar solvent.

(Peptide Containing Three or More Amino Acids)

The peptide used in the present invention is not limited by the type of amino acids and the combination thereof, and any peptide can be used as long as the peptide contains three or more amino acids. The peptide may be a protein derived from an animal or a plant and a decomposition product thereof, or may be a peptide obtained by organic synthesis.

Examples of the protein derived from an animal or a plant and the decomposition product thereof include animal proteins derived from meats of animals such as bovine, swine, and poultry, fishes, animal milks, eggs, and the like; plant proteins derived from soybean, carrot, wheat, corn, pea, and the like; and decomposition products thereof. In particular, peptides derived from soybean, collagen, and carrot are preferable, and a decomposition product of soybean, a collagen peptide, and a peptide derived from carrot are most preferable. As the protein, a physically ground raw material or an extract from the raw material may be used. Extraction can be done with a solvent such as water or an organic solvent. The decomposition products of the above-described proteins can be obtained by degradating the ground products or extracts of the above-described animal and plant proteins with an acid, alkali, or enzyme.

Collagen is the main protein constituting the connective tissue in animals and contained in a large amount in bone, tendons, skin, blood vessel walls, and the like. A molecule of collagen has one or more triple helix structures, the constituent polypeptide chains having different amino acid sequences. Gelatin, which is a denatured collagen, is a water-soluble protein having a molecular weight of about 300,000 to several tens of thousands and obtained by extracting a collagen-containing raw material with warm (hot) water. Alkali-treated gelatin (having an isoelectric point of 4.8 to 5.3) and acid-treated gelatin (having an isoelectric point of 7 to 9) are included.

The collagen peptide is obtained by, for example, decomposing the above-described collagen or gelatin as follows. First, the skin or bone of bovine, swine, or the like is subjected to an alkali treatment in which the skin or bone is immersed in an alkali solution for 2 to 3 months or an acid treatment in which the skin or bone is immersed in a dilute hydrochloric acid or the like for a short period of time, as a pretreatment for removing impurities contained in a raw material and facilitating extraction. For example, when the raw material is bovine bone, inorganic substances such as calcium phosphate are contained in the bone, so that the bone is preliminarily immersed in a dilute hydrochloric acid to remove the inorganic substances, and then subjected to extraction with warm (hot) water to obtain gelatin. In the extraction with warm (hot) water, generally, the first extraction temperature is 50 to 60° C., and the extraction temperature is increased gradually, from the second time to a final point where the water used for extraction is boiled. Next, the resultant gelatin is hydrolyzed with a commonly used acid or enzyme. In this manner, a collagen peptide can be obtained. Such a collagen peptide can be obtained easily or is commercially available. Examples thereof include Nippi Peptide PBF and Nippi Peptide PRA (both manufactured by Nippi, Inc.), SCP-5000 and SCP-3100 (both manufactured by Nitta Gelatin Inc.), Collagen Peptide DS (manufactured by Kyowa Hi Foods Co., Ltd.), and Pharconix CTP (manufactured by ICHIMARU PHARCOS CO., LTD.).

The decomposition product of soybean is obtained by, for example, defatting soybeans, extracting the defatted soybeans with water to obtain soybean milk, acid-precipitating the soybean milk to obtain a soy protein isolate, decomposing the soy protein isolate by a hydrolysis treatment with an alkali, an acid, enzyme, oxidizing agent, or reducing agent, or a combination thereof. If necessary, peptides having a certain molecular weight can be fractionated. Decomposition products of soybean are commercially available. For example, Hinute S, R, D1, D3, DC5, SMS, and SMP (all of which are manufactured by Fuji Oil Co., Ltd.) are included.

Since a peptide derived from carrot has a similar amino acid composition to that of animal collagen, it can be used preferably. For example, a peptide derived from Daucus carota L. is a collagen-like peptide.

There is no particular limitation on the average molecular weight of the peptide containing three or more amino acids. Preferably, the average molecular weight is 7,000 or less, more preferably 400 to 7,000, even more preferably 400 to 6,500, particularly preferably 400 or more and less than 3,000. The use of such a peptide containing three or more amino acids can be expected to enhance the bioactivities of proanthocyanidins. If the average molecular weight is more than 7,000, then the peptide binds to many proanthocyanidins, and thus a large conjugate is formed, thereby precipitation or suspension may be caused. Furthermore, proanthocyanidins may be prevented from being absorbed in the body. A peptide having an average molecular weight of less than 400 may cause aggregation and precipitation in a long-term storage.

(Water-Soluble Conjugate)

The water-soluble conjugate of the present invention consists of proanthocyanidins and a peptide containing three or more amino acids, in particular, proanthocyanidins in a plant extract is preferably used. There is no particular limitation on the ratio between the proanthocyanidins and the peptide containing three or more amino acids. In view of the protection of the proanthocyanidins stably in the water-soluble conjugate, it is preferable that the peptide containing three or more amino acids, e.g., a peptide derived from soybean or collagen, is contained in an amount of one part by weight or more, more preferably 5 parts by weight or more, even more preferably 5 parts by weight to 300 parts by weight, most preferably 5 parts by weight to 150 parts by weight, with respect to 1 part by weight of proanthocyanidins. If the amount of the peptide containing three or more amino acids is less than one part by weight, then the protection of the proanthocyanidins stably in the conjugate may not be sufficient.

It is preferable that the water-soluble conjugate of the present invention is manufactured specifically by mixing proanthocyanidins and a peptide containing three or more amino acids in a polar solvent such as water or ethanol, in particular, proanthocyanidins in a plant extract is used. Water is preferable as the polar solvent. Mixing can be performed, for example, by mixing a proanthocyanidin-containing solution obtained by dissolving a dry powder of proanthocyanidins in a polar solvent and a peptide-containing solution obtained by dissolving a peptide containing three or more amino acids in the same polar solvent; by adding a dry powder of proanthocyanidins to a peptide-containing solution; by adding a dry powder of a peptide to a proanthocyanidin-containing solution; or by mixing a dry powder of proanthocyanidins and a dry powder of a peptide and then adding a polar solvent thereto.

When a proanthocyanidin-containing solution and a peptide-containing solution are mixed, there is no particular limitation on the mixing ratio between these solutions. In view of obtaining a water-soluble conjugate without causing precipitation, the amount of the proanthocyanidin-containing solution is preferably 1/50 parts by volume or more, more preferably 1/10 parts by volume or more, with respect to one part by volume of the peptide-containing solution.

When mixing is performed by adding a dry powder of proanthocyanidins to a peptide-containing solution or by adding a dry powder of a peptide to a proanthocyanidin-containing solution, it is preferable to add the dry powder while stirring or to add the dry powder and then stir the resultant mixture, in view of preventing precipitation.

When a dry powder of proanthocyanidins and a dry powder of a peptide are mixed and then a polar solvent is added thereto, it is preferable to spray the polar solvent while stirring the mixed powder in view of obtaining a homogeneous water-soluble conjugate.

The obtained water-soluble conjugate may be then pulverized by a processing method usually used by those skilled in the art. Pulverization can be performed, for example, by using a fluidized bed granulator or the like and by spraying a polar solvent while stirring the mixed powder of proanthocyanidins and the peptide. Even when pulverized, the pulverized water-soluble conjugate has the same effects as the water-soluble conjugate in a liquid form, and, for example, proanthocyanidins suffer less degradation, e.g., discoloration, have retained or enhanced bioactivities, and can exhibit the bioactivities in the body. Furthermore, it is preferable to pulverize the water-soluble conjugate and dissolve the pulverized water-soluble conjugate again in a solvent such as water at the time of use because degradation of proanthocyanidins, e.g., discoloration, is small.

In the water-soluble conjugate of the present invention, a peptide containing three or more amino acids is bound to proanthocyanidins, in particular, proanthocyanidins in a plant extract when the plant extract is used, and thus, the protective stability of proanthocyanidins can be increased and also bioactivities thereof can be retained or increased. Therefore, precipitation does not occur even in a long-term storage in a liquid state, and the water-soluble conjugate can be transported into the body while maintaining those bioactivities. The water-soluble conjugate of the present invention can be utilized as food products, drugs, quasi-drugs, cosmetics, toiletries, and the like, and the effects of the water-soluble conjugate can be exhibited by the use thereof for either of the purposes of oral administration and percutaneous administration. In particular, the water-soluble conjugate of the present invention is suitably utilized as an oxidation stabilizer and the like for liquid products and the like.

(Composition Containing Water-Soluble Conjugate)

The composition of the present invention contains the above-described water-soluble conjugate, and may contain other components, if necessary. This composition can be utilized as food products, drugs, quasi-drugs, cosmetics, toiletries, and the like as is the case with the above-described water-soluble conjugate.

The amount of the water-soluble conjugate that is contained in the composition of the present invention varies depending on the administration method and the dosage form, and is not limited. Preferably, the water-soluble conjugate is contained in the composition so that the amount of proanthocyanidins is 0.00001 wt % to 50 wt %, more preferably 0.001 wt % to 40 wt %, even more preferably 0.01 wt % to 20 wt %, expressed in terms of dry weight.

The composition of the present invention may contain other components usually used for drugs, food products, quasi-drugs, cosmetics, and the like in addition to the above-described water-soluble conjugate, as long as the effects of the composition are not impaired. Examples of such components include water, a medicinal component, an antioxidant, and an oil, a moisturizing agent, a surfactant, an ultraviolet absorber, an absorption promoter, a flavor, a pigment, a preservative, a thickener, a chelating agent, and an antiseptic and antifungal agent.

Examples of the medicinal component include an active oxygen remover, an antioxidant, an antiphlogistic and analgesic agent, an antihistamic agent, an antipruritic agent, a disinfectant, a vitamin preparation, and a hormone preparation.

The antioxidant is added in order to further increase the stability of proanthocyanidins to oxidation. Furthermore, it prevents oxidation of proteins and lipids in the body, and effects of improving and protecting the properties of skin also can be obtained. Examples of the antioxidant include carotenoids such as vitamin A, vitamin Bs, ascorbic acid, vitamin E, derivatives thereof or salts thereof, L-cysteines and derivatives thereof and salts thereof, riboflavin, SOD, mannitol, tryptophan, histidine, quercetine, gallic acid and derivatives thereof, a tea extract, and extracts such as a glutathione yeast extract. Ascorbic acid and derivatives thereof and salts thereof are preferable.

Ascorbic acid or a derivative thereof or a salt thereof (hereinafter referred to as “ascorbic acid or the like”) not only increases the stability of proanthocyanidins, but also synergistically exhibits an effect on the skin and the lipid metabolism and increases the effect of improving the properties of the skin (e.g., an effect of improving the vitality and the luster of the skin) and the effect of protecting blood vessels even more. Ascorbic acid or the like can be contained in an amount of preferably 0.1 parts by weight to 50 parts by weight, more preferably 0.2 parts by weight to 20 parts by weight, with respect to 1 part by weight of proanthocyanidins.

When the composition of the present invention is utilized for the purpose of oral administration of a food product or the like, the composition can be further mixed with an additive agent such as an excipient, an expander, a binder, a thickener, an emulsifier, a coloring agent, a flavor, a food additive, and a seasoning agent, if necessary. For example, the composition can be mixed with a food additive serving as a nutritional supplement, such as royal jelly, vitamins, proteins, calcium substances such as eggshell calcium, chitosan, lecithin, chlorella powder, Angelica keiskei powder, and Corchorus olitorius powder; and a seasoning agent, such as stevia powder, ground green tea powder, lemon powder, honey, reducing maltose, lactose, and a sugar solution. This composition can be shaped into capsules such as soft capsules and hard capsules, tablets, pills, or the like, or into the form of powder, granule, tea, tea bags, candy, liquid, paste, or the like. Depending on the shape or the individual's preference, the resultant products may be eaten or drunk as they are, or may be dissolved in water, hot water, milk, or the like for drinking.

The composition of the present invention can be used as drugs, quasi-drugs, cosmetics, and toiletries for the purpose of percutaneous administration. For example, the composition can be used as a skin lotion, a facial cream, a milky lotion, a cream, a pack, a hair tonic, a hair cream, a shampoo, a hair rinse, a hair treatment, a body shampoo, a facial cleanser, a soap, a foundation, a face powder, a lipstick, a lip gloss, a rouge, an eye shadow, a hairdressing, a hair restorer, a hydrophilic ointment, an hydrophobic ointment, an eyedrop, an eyewash, a dentifrice, a mouthwash, a poultice, and a gel. Moreover, topical, long-term administration of the composition is also possible by a method, for example, of allowing a carrier, such as a poultice or a gel, or a crosslinking agent to carry or absorb the composition, and pasting the carrier or the crosslinking agent on an affected part.

There is no particular limitation on the daily dosage when the composition of the present invention is orally administered. However, when the daily dosage is within a range of preferably 0.02 g to 1 g expressed in terms of proanthocyanidins, various bioactivities can be obtained. In the case of percutaneous administration, the composition of the present invention is administered topically, so that the bioactivities can be obtained as long as a product is prepared so as to contain proanthocyanidins at the above-described specific concentration.

When an appropriate amount of the composition of the present invention is taken, a superior antioxidation effect in blood, a superior effect of improving the blood flow, a superior effect of improving the properties of the skin, and the like can be obtained compared to those when proanthocyanidins or a peptide containing three or more amino acids are taken independently. In particular, when a plant extract containing 20 wt % or more OPCs expressed in terms of dry weight is used, particularly superior effects can be obtained.

### EXAMPLES

Hereinafter, the present invention will be described by means of examples. However, it should be appreciated that the present invention is not limited to these examples.

### Example 1

### Water-Soluble Conjugate

First, 0.5 mL of an aqueous solution containing 0.2 wt % of a pine bark extract (dimer to tetramer: 40 wt %, pentamer or greater: 25.1 wt %, catechin: 5.1 wt %, trade name: Flavangenol, TOYO SHINYAKU Co., Ltd.) and 0.5 mL of an aqueous solution containing 4 wt % of a decomposition product of soybean (average molecular weight about 400, Hinute PM, Fuji Oil Co., Ltd.) were mixed to obtain a transparent mixture. Whether or not a conjugate was formed was determined by detecting catechins, dimeric OPCs, and trimeric OPCs in this mixture by silica gel chromatography (TLC). That is to say, for the mixture, when spots does not appear at the same positions as standards of the above-described components usually contained in the pine bark extract, it is determined that the above-described components are used to form a conjugate. On the other hand when the spots appears, it is determined that a conjugate is not formed. The results are shown in Table 1. TLC was performed under the following conditions, and standards of catechin (Rf value: 0.8) and dimeric and trimeric OPCs (dimer: proanthocyanidin B-2 (Rf value: 0.6), trimer: proanthocyanidin C-1 (Rf value: 0.4)) were used as standard indicators:

TLC: Silica gel plate (manufactured by Merck & Co., Inc.)

Eluent: Benzene/Ethyl formate/Formic acid (2/7/1)

Detection reagent: Sulfuric acid and Anisaldehyde sulfate

Amount of sample: 10 μL each

### Comparative Examples 1 to 3

Whether or not a conjugate was formed was determined by TLC in the same manner as in Example 1, except that an aqueous solution containing 0.1 wt % of the pine bark extract was used instead of the mixture in Example 1 (Comparative Example 1). In the same manner as described above, whether or not a conjugate was formed was determined by TLC also for an aqueous solution containing 2 wt % of the decomposition product of soybean (average molecular weight about 400) in Example 1 (Comparative Example 2) and for an aqueous solution containing 2 wt % of glycine (molecular weight 75: manufactured by Wako Pure Chemical Industries, Ltd.) (Comparative Example 3). The results are shown in Table 1 together.

TABLE 1

| | | Standard | | |
|---|---|---|---|---|
| | | Catechin | Dimer | Trimer |
| Example 1 | Pine bark extract | + | − | − |
| | Decomposition product | | | |
| | of soybean | | | |
| Comparative | Pine bark extract | + | + | + |
| Example 1 | | | | |
| Comparative | Decomposition product | − | − | − |
| Example 2 | of soybean | | | |
| Comparative | Amino acid | − | − | − |
| Example 3 | | | | |

+ indicates that the spot was detected at the same position as the standard.
− indicates that the spot was not detected at the same position as the standard.

As can be seen from the results of Example 1 in Table 1, catechin was detected, but proanthocyanidins (OPCs) having a degree of polymerization of 2 and 3 were not detected from the mixture of the pine bark extract and the decomposition product of soybean in Example 1. In contrast, both of catechin and proanthocyanidins (OPCs) having a degree of polymerization of 2 and 3 were detected from the pine bark extract-containing solution in Comparative Example 1, and neither of them were detected from the solution containing the decomposition product of soybean in Comparative Example 2. Thus, it is found that when a pine bark extract and a decomposition product of soybean are mixed, proanthocyanidins (OPCs) having a degree of polymerization of 2 and 3, which are contained in the pine bark extract, are no longer detected. It is apparent that this is because proanthocyanidins having a degree of polymerization of 2 or more in the pine bark extract and the decomposition product of soybean form a conjugate that is soluble in water.

### Example 2

### Evaluation of Aggregation and Precipitation

(1) Preparation of Proanthocyanidin-Containing Solutions

First, 20 g of the pine bark extract (hereinafter referred to as the “pine bark extract A”) used in Example 1 were separated using Sephadex LH-20 (manufactured by Pharmacia Biotech, Inc.) to collect 7.6 g of proanthocyanidins having a degree of polymerization of 2 to 4 and 5.1 g of proanthocyanidins having a degree of polymerization of 5 or more expressed in terms of dry powder weight. It should be noted that separation with Sephadex LH-20 was performed twice under the following conditions.

First, Sephadex LH-20 swollen with water was packed in a 50×500 mm column to a column volume of 500 mL, and washed with 500 mL of ethanol. Then, 10 g of the above-described pine bark extract were dissolved in 200 mL of ethanol, and this solution was applied on the column for adsorption, and then eluted gradiently using 100 to 80% (v/v) ethanol-water mixed solvents, and the resultant eluate was collected in fractions of 100 mL each. Each of the fractions was subjected to silica gel chromatography (TLC) to detect whether or not OPCs were eluted, using standards of dimeric to tetrameric OPCs (dimer: proanthocyanidin B-2 (Rf value: 0.6), trimer: proanthocyanidin C-1 (Rf value: 0.4), and tetramer: cinnamtannin A₂ (Rf value: 0.2)) as standard indicators. TLC was performed under the same conditions as in Example 1.

The fractions in which OPCs were detected were combined, and freeze-dried to obtain a powder. Next, 1000 mL of a 50% (v/v) water-acetone mixture were applied on the column in which elution of OPCs were not detected to elute proanthocyanidins having a degree of polymerization of 5 or more, and the collected fractions were freeze-dried to obtain a powder.

Then, 0.1 g of the obtained proanthocyanidins having a degree of polymerization of 5 or more and 1 g of a powder of the above-described pine bark extract A were mixed to prepare a pine bark extract (hereinafter referred to as the “pine bark extract B”) containing 36 wt % of dimer to tetramer (OPCs) and 31 wt % of proanthocyanidins having a degree of polymerization of 5 or more.

The pine bark extract B obtained as described above was dissolved in water so that the final concentration was 0.2 wt % (this solution was taken as the proanthocyanidin-containing solution 1). Separately, the above-described OPC fraction of the pine bark extract A was made into a proanthocyanidin-containing solution 2, and the above-described fraction of proanthocyanidins having a degree of polymerization of 5 or more was made into a proanthocyanidin-containing solution 3.

(2) Preparation of Solutions Containing Peptide and Amino Acid

Aqueous solutions of 3 mL each were prepared using collagen (average molecular weight 300,000: manufactured by KOKEN CO., LTD.), Nippi Peptide PA-100 (average molecular weight 10,000: manufactured by Nippi, Inc.), Collagen Peptide DS (average molecular weight 7,000: manufactured by Kyowa Hi Foods Co., Ltd.), SCP-5000 (average molecular weight 5,000: manufactured by Nitta Gelatin Inc.), Pharconix CTP (average molecular weight 3,000: manufactured by ICHIMARU PHARCOS CO., LTD.), Nippi Peptide PA-10 (average molecular weight 1,000: manufactured by Nippi, Inc.), a decomposition product of soybean (average molecular weight about 400, Hinute PM, manufactured by Fuji Oil Co., Ltd.), a dipeptide of serine-glutamic acid (Sigma-Aldrich Japan KK), and glycine (molecular weight 75: manufactured by Wako Pure Chemical Industries, Ltd.) so that the aqueous solutions contained 10.0 wt % of the above-mentioned collagen, collagen peptide, or amino acid.

(3) Evaluation

First, 1 mL each of the above-described proanthocyanidin-containing solutions 1 to 3 were independently mixed with 1 mL each of the peptide or amino acid solutions prepared as described above. These mixtures were allowed to stand at room temperature for one week, and then, whether or not precipitation and suspension occurred was observed visually. Moreover, these mixtures were also allowed to stand at 40° C. for 6 months, and then whether or not precipitation and suspension occurred was observed visually. The results are shown in Table 2. As controls, a solution obtained by adding 1 mL of water to 1 mL of the proanthocyanidin-containing solution 1 (control 1) and a solution obtained by mixing 1 mL of an ascorbic acid-containing solution (0.1 wt %) with 1 mL of the proanthocyanidin-containing solution 1 (control 2) were prepared, and whether or not precipitation and suspension occurred was observed visually in the same manner as described above.

TABLE 2

| | | | Amino acid, peptide, etc. | | | | | | | | | | |
|---|---|---|---|---|---|---|---|---|---|---|---|---|---|
| | | | | Dipeptide | Decompo- | | | | | | | | |
| | | | | of serine- | sition | | | | | | | | |
| | | | | glutamic | product | | | | | | | | |
| | | | Glycine | acid | of soybean | Collagen peptide | | | | | Collagen | None | V.C |
| | | | 75 | 234 | 400 | 1,000 | 3,000 | 5,000 | 7,000 | 10,000 | 300,000 | — | — |
| Proanthocyanidin- | Room | Precipitation | − | − | − | − | − | − | − | + | + | − | − |
| containing solution 1 | temperature | Suspension | − | − | − | − | − | − | ± | + | − | − | − |
| (Dimer to tetramer: | 1 week | | | | | | | | | | | | |
| 0.07 wt %, Pentamer | 40° C. | Precipitation | + | + | − | − | − | − | − | + | + | + | + |
| or more: 0.06 wt %) | 6 months | Suspension | + | + | − | − | − | − | ± | + | − | + | + |
| Proanthocyanidin- | Room | Precipitation | − | − | − | − | − | − | − | + | + | | |
| containing solution 2 | temperature | Suspension | − | − | − | − | − | − | − | + | − | | |
| (Dimer to tetramer) | 1 week | | | | | | | | | | | | |
| | 40° C. | Precipitation | + | + | − | − | − | − | − | + | + | | |
| | 6 months | Suspension | + | + | − | − | − | − | − | + | − | | |
| Proanthocyanidin- | Room | Precipitation | − | + | + | + | + | + | + | + | + | | |
| containing solution 3 | temperature | Suspension | − | + | + | + | + | + | + | + | − | | |
| (Pentamer or more) | 1 week | | | | | | | | | | | | |
| | 40° C. | Precipitation | + | + | + | + | + | + | + | + | + | | |
| | 6 months | Suspension | + | + | + | + | + | + | + | + | − | | |

+: Precipitation was observed.
±: Precipitation was observed slightly.
−: Precipitation was not observed.
V.C . . . Ascorbic acid

As can be seen from Table 2, when the mixtures were stored at room temperature for one week, nearly no suspension was observed in the solutions prepared by mixing proanthocyanidins having a degree of polymerization of 2 to 4 (proanthocyanidin-containing solution 2) or proanthocyanidins containing OPCs (proanthocyanidin-containing solution 1) with amino acid (glycine) and the peptides having two or more amino acids (the serine-glutamic acid dipeptide, the decomposition product of soybean, and the collagen peptides having an average molecular weight of 1,000, 3,000, 5,000, and 7,000), water alone (control 1), or ascorbic acid (control 2). However, among these mixtures, when the mixtures were stored at 40° C. for 6 months, precipitation or suspension was observed in the mixtures of the above-described proanthocyanidin-containing solutions with amino acid (glycine), the peptide containing not more than two amino acids (the serine-glutamic acid dipeptide), water alone (control 1), or ascorbic acid (control 2). Moreover, when the collagen having an average molecular weight of 300,000 was used, a gelled solid was precipitated. It is found that among the conjugates of the present invention of proanthocyanidins and a peptide containing three or more amino acids (i.e., having an average molecular weight of 400 or more), a water-soluble conjugate shows a superior storage stability and tends not to cause precipitation and suspension.

### Example 3

### Effect of Protecting Proanthocyanidins

Food products (granular products) were produced from dry powders of the pine bark extract A used in Example 1, the dipeptide of serine-glutamic acid and the decomposition product of soybean used in Example 2, and a soybean protein (having an average molecular weight of 10,000 or more, manufactured by Fuji Oil Co., Ltd.) that was extracted from soybean and that was not subjected to a decomposition treatment, by using a fluidized bed granulator while spraying water (food products 1 to 4). The amount of each component is shown below.

TABLE 3

| | Food | Food | Food | Food |
|---|---|---|---|---|
| | product | product | product | product |
| | 1 | 2 | 3 | 4 |
| Pine bark extract | 10 | 10 | 10 | 10 |
| (Containing Dimer to tetramer: | | | | |
| 40 wt %, Pentamer or more: | | | | |
| 25 wt %) | | | | |
| Dipeptide of serine-glutamic | — | — | 50 | — |
| acid (Molecular weight 234) | | | | |
| Decomposition product of | 50 | — | — | — |
| soybean (Average molecular | | | | |
| weight 400) | | | | |
| Soybean peptide | — | 50 | — | — |
| (Average molecular weight | | | | |
| 10,000 or more) | | | | |
| Reducing maltose | 40 | 40 | 40 | 90 |
| (Additive) | | | | |

Values are expressed in terms of parts by weight.

Rats (Charles River Laboratories Japan, Inc.) at the age of 5 weeks were given a common pellet diet (MF; Oriental Yeast Co., Ltd.) for one week for acclimation, and then blood was collected from the ocular fundus of the rats, and the SOD activity in blood was measured using a measuring kit (SOD Test Wako: Wako Pure Chemical Industries, Ltd.). After the measurement, the rats were divided into a total of 5 groups of 6 each so that the average value of the SOD activity was uniform among the groups. Separately, the respective above-described food products 1 to 4 were mixed with purified water so that the final concentration of the pine bark extract was 10 mg/mL (test solutions 1 to 4). These mixtures were orally administered to the rats in the respective groups using a sonde so that the pine bark extract contained in these mixtures was administered at 100 mg/kg body weight. As a control, purified water was administered to one group. After 45 minutes and after 90 minutes from the oral administration, blood was collected from the ocular fundus again, and the SOD activity was measured. The results are shown in Table 4.

TABLE 4

| | | Time after administration (min) | |
|---|---|---|---|
| | Conponent | 45 | 90 |
| Test solution 1 | Food product 1 | 2.36 ± 0.48 | 4.16 ± 0.80 |
| Test solution 2 | Food product 2 | 1.13 ± 0.22 | 2.78 ± 0.43 |
| Test solution 3 | Food product 3 | 1.68 ± 0.54 | 2.50 ± 0.44 |
| Test solution 4 | Food product 4 | 1.75 ± 0.44 | 2.46 ± 0.71 |
| Purified water | None | 1.45 ± 0.19 | 1.98 ± 0.15 |

Mean ± Standard error

From the results in Table 4, it can be seen that with the test solution 1 (containing the food product 1 in which the pine bark extract and the decomposition product of soybean were used), the SOD activity in blood was increased more than with the test solution 2 (containing the food product 2 in which the pine bark extract and the soybean protein were used), the test solution 3 (containing the food product 3 in which the pine bark extract and the dipeptide of serine-glutamic acid were used), the test solution 4 (containing the food product 4 in which only the pine bark extract was used), or purified water (containing no food product). This indicates that in the test solution 1, the antioxidative activity, which is one of the bioactivities of proanthocyanidins, was at least protected from the digestion process, and proanthocyanidins were absorbed, so that the SOD activity in the body due to proanthocyanidins was enhanced. Moreover, it is apparent that absorption was inhibited in the test solution 2 containing the food product 2 in which the pine bark extract and the soybean protein were used, because the SOD activity after 45 minutes from ingestion was particularly low.

### Example 4

### Production of a Food Composition

A drink (500 mL) was produced using the following amounts of components:

| | | |
|---|---|---|
| Soybean peptide (average molecular weight 700) | 0.1 | wt % |
| Pine bark extract | 0.02 | wt % |
| Sodium ascorbate | 0.016 | wt % |
| Fructose | 0.3 | wt % |
| Orange juice | 1.0 | wt % |

| | |
|---|---|
| Water | q.s. |

### Example 5

### Production of an External Preparation)

An external preparation (skin lotion) was prepared using the following amounts of components. This skin lotion provided a superior effect of improving epidermal blood flow and a superior moisture retaining property when applied to the skin, and was a stable external preparation in which no precipitation occurred:

| | | |
|---|---|---|
| Collagen peptide (average molecular weight 1,000) | 0.05 | wt % |
| Pine bark extract | 0.05 | wt % |
| Ethyl alcohol | 20 | wt % |
| 1,3-butylene glycol | 10 | wt % |
| Allantoin | 0.2 | wt % |

| | |
|---|---|
| Water | q.s. |

### INDUSTRIAL APPLICABILITY

The water-soluble conjugate of the present invention consists of proanthocyanidins and a peptide containing three or more amino acids. With this water-soluble conjugate, the protective stability of proanthocyanidins can be increased, and also the bioactivities of proanthocyanidins can be retained or increased. Thus, the water-soluble conjugate does not cause precipitation even when stored for a long term in a liquid state, and can be transported into the body while maintaining the bioactivities. The water-soluble conjugate of the present invention is utilized as food products, drugs, quasi-drugs, cosmetics, toiletries, and the like, and used for either of the purposes of oral administration and percutaneous administration. In particular, it can be utilized as an oxidation stabilizer for liquid products and the like.

1. A water-soluble conjugate consisting of a proanthocyanidin and a peptide containing three or amino acids.

2. The conjugate of claim 1, wherein the average molecular weight of the peptide is not more than 7,000.

3. The conjugate of claim 1, wherein the proanthocyanidin is derived from a plant extract.

4. A composition comprising the water-soluble conjugate of claim 1.

5. The conjugate of claim 2, wherein the proanthocyanidin is derived from a plant extract.

6. A composition comprising the water-soluble conjugate of claim 2.

7. A composition comprising the water-soluble conjugate of claim 3.
